(19) United States
(12) Patent Application Publication
(10) Pub. No.: US 2025/0108259 A1
(43) Pub. Date: Apr. 3, 2025

(54) Exercise Tracking Prediction Method

(71) Applicant: Apple Inc., Cupertino, CA (US)

(72) Inventors: Stefano Alletto, Mountain View, CA (US); Abhishek Narain, San Ramon, CA (US); Denis Tome', Sunnyvale, CA (US)

(21) Appl. No.: 18/823,601

(22) Filed: Sep. 3, 2024

Related U.S. Application Data
(60) Provisional application No. 63/586,610, filed on Sep. 29, 2023.

(51) Int. Cl. A63B 24/00

(52) U.S. Cl. CPC A63B 24/0062; A63B 24/0006; A63B 2024/0068; A63B 2220/05

(57) ABSTRACT

Predicting and counting repetitions of a physical activity includes capturing image data of a body in motion, and determining, based on a first set of frames of the image data, one or more confidence values for one or more motion classes. In response to receiving an additional frame of the image data, the one or more confidence values for the one or more motion classes are revised. In response to determining that the confidence values for at least one of the one or more motion classes satisfies a stability threshold, the at least one of the one or more motion classes is assigned to the body in motion. In response to a determination that the repetition has ended, a repetition count for the at least one of the one or more motion classes is modified.

### BACKGROUND

Current techniques in image data analysis provide for numerous insights into a scene depicted in an image. For example, object detection can be used to identify objects in a scene, or characteristics of an object in a scene. One application is to apply image data to a network to determine a pose of a person.

Shortfalls exist when it comes to predicting motion of an object. For example, in order to predict an activity undertaken by a person, a video sequence of frames may be fed into a network, and a prediction for the video sequence may be obtained based on the entirety of the video. Problems exist in obtaining real time predictions for a user activity.

### BRIEF DESCRIPTION OF THE DRAWINGS

FIGS. 1A-1B show example diagrams of a technique for predicting a user activity, according to one or more embodiments.

FIG. 2 shows a flowchart of a technique for counting repetitions of predicted user activities, according to one or more embodiments.

FIG. 3 shows, in flowchart form, a technique for determining confidence values for motion classes, in accordance with one or more embodiments.

FIG. 4 shows a diagram of a technique for counting repetitions of predicted user activities, in flowchart form, a technique for applying a visual treatment to image content, according to one or more embodiments.

FIG. 5 shows an example system diagram of an electronic device, according to one or more embodiments.

FIG. 6 shows, in block diagram form, a simplified multifunctional device according to one or more embodiments.

### DETAILED DESCRIPTION

This disclosure is directed to systems, methods, and computer readable media for exercise tracking and prediction. In general, techniques disclosure are directed to capturing image data of the body of the motion and, in real time, predicting an activity being performed by a user. In addition, techniques described herein are directed to managing repetition count for the activity being performed.

According to one or more embodiments, image data can be captured of a user performing an activity, such as an exercise. Although the exercise may not be known to the system, the system can make a prediction as to which activities they performed while the activity is in progress. Generally, a network may be trained to ingest image data, determined body pose information, and based on body pose information, make the prediction as to an activity being performed. The network may be trained to predict the activity being performed based on body pose in a current frame, as well as prior frames. Prediction information may be generated by the network, for example on a frame-by-frame basis, for each of the set of user activities. As a prediction information stabilizes over time, at least one of the set of activities can be identified of the activity be performed in the image data.

In some embodiments, the network may predict, based on the pose information, an initialization and duration of the corresponding activity. That is, while the activity is in progress, a prediction can be made as to the end of the activity, thereby allowing prediction to be made in real time without having image data of the full activity, which would normally be available in an offline mode but is not available when performing the predictions in real time. The initialization and duration predictions can be plotted to detect heatmaps. The heatmaps may be used to determine whether the prediction information for a particular class of activity is stabilized over time. That is, the confidence values for each class can be updated over a series of frames to consider the most recently processed frame, along with prior frames, to refine the confidence values. The predicted activity is held until the end of the predicted duration, or until the identified activity is complete. Upon completion of the activity, a repetition will be counted for the activity. Feedback regarding the repetition may be provided, for example, via a user interface for presentation to the user.

In one or more embodiments, once a repetition is complete, the confidence values for each class of activity may be reset. However, in some embodiments, the class of activity identified in prior repetition may be weighted for the next repetition to enhance prediction of repeated motions such as exercises or other repetitive activities.

In the following description, for purposes of explanation, numerous specific details are set forth in order to provide a thorough understanding of the disclosed concepts. As part of this description, some of this disclosure's drawings represent structures and devices in block diagram form in order to avoid obscuring the novel aspects of the disclosed embodiments. In this context, it should be understood that references to numbered drawing elements without associated identifiers (e.g., 100) refer to all instances of the drawing element with identifiers (e.g., 100a and 100b). Further, as part of this description, some of this disclosure's drawings may be provided in the form of a flow diagram. The boxes in any particular flow diagram may be presented in a particular order. However, it should be understood that the particular flow of any flow diagram is used only to exemplify one embodiment. In other embodiments, any of the various components depicted in the flow diagram may be deleted, or the components may be performed in a different order, or even concurrently. In addition, other embodiments may include additional steps not depicted as part of the flow diagram. The language used in this disclosure has been principally selected for readability and instructional purposes and may not have been selected to delineate or circumscribe the disclosed subject matter. Reference in this disclosure to “one embodiment” or to “an embodiment” means that a particular feature, structure, or characteristic described in connection with the embodiment is included in at least one embodiment, and multiple references to “one embodiment” or to “an embodiment” should not be understood as necessarily all referring to the same embodiment or to different embodiments.

It should be appreciated that in the development of any actual implementation (as in any development project), numerous decisions must be made to achieve the developers' specific goals (e.g., compliance with system and business-related constraints), and that these goals will vary from one implementation to another. It will also be appreciated that such development efforts might be complex and time consuming but would nevertheless be a routine undertaking for those of ordinary skill in the art of image capture having the benefit of this disclosure.

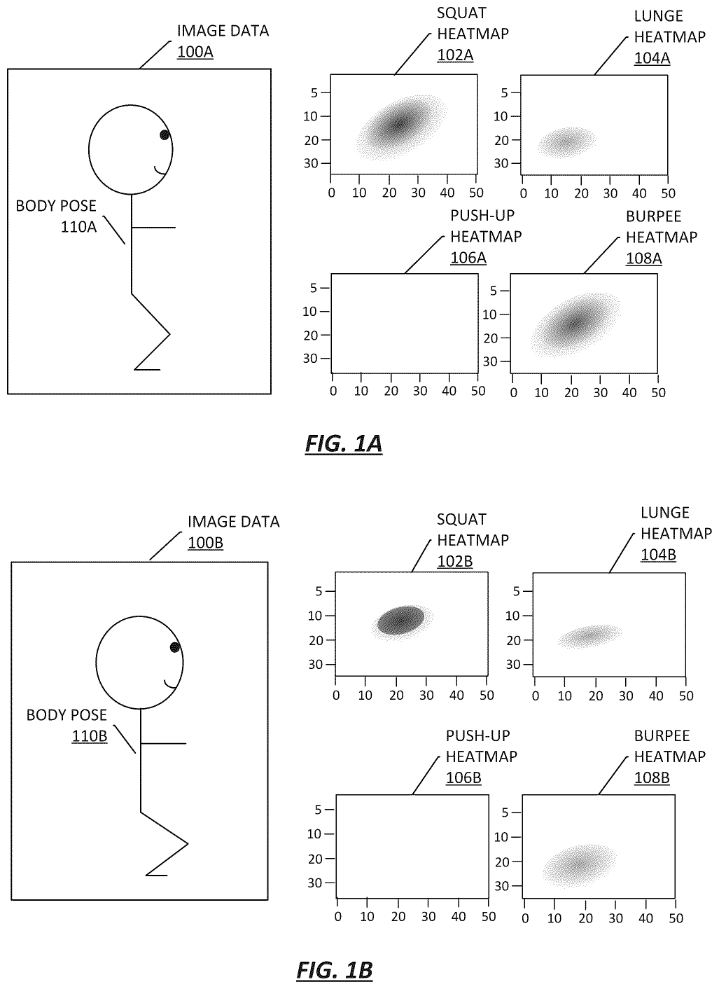

Referring to FIG. 1A, a diagram is presented in which image data is processed to make the prediction as to the user activity being performed in the image data. According to one or more embodiments, the image data 100A may be captured by an electronic device. Electronic device may be any kind of device that includes a camera or other sensors from which pose information can be detected for a person in the invention. The electronic device capturing the image data may be the same or different from an electronic device performing the prediction of the activity.

In some embodiments, image data 100A may be applied to a network trained to predict a body pose present in the image. Body pose may be predicted, for example, in the form of a 2D pose, a 3D pose, or the like. Body pose may include, for example a classification of a pose, a representative skeleton for the pose, or the like. In the image data 100A, a person is shown in body pose 110A, performing a light bend of the knee.

According to one or more embodiments, a same or different network may be trained to predict future achievable results from a current pose. In some embodiments, the network may consider the current body pose, along with body poses from prior frames. The network may be trained for a predefined set of activities, such as exercises or other repeatable activities. Confidence values are determined for each of these activities based on the current pose and/or prior pose data. The confidence values indicate a likelihood of an outcome for each of the set of activities.

For purposes of the example of FIG. 1A, four potential exercises are considered by the network. These include a squat, a lunge, a push-up, and a burpee. For each activity, a set of confidence values for potential outcomes are plotted to generate a heatmap. In this example, confidence values are determined and plotted for a squat heatmap 102A, a lunge heatmap 104A, a push-up heatmap 106A, and a burpee heatmap 108A. Each of the diagrams depicts a graph in which the x-axis indicates a frame or time at which the activity is predicted to be initiated. The y-axis indicates a predicted duration of the activity. Thus, the confidence values of squat heatmap 102A indicate the likelihood for each potential outcome of the squat (e.g., each potential initiation and duration). In the example of FIG. 1A, squat heatmap 102A depicts the most defined heatmap, while burpee heatmap 108A is similarly defined. Lunge heatmap 104A depicts a small scattering of confidence values, indicating that a lunge is still possible outcome from current body pose 110A. Notably, push-up heatmap 106A is blank because, while the body pose 110A is in a standing position, a push-up does not include a standing position. As such, no potential outcomes exist for a push-up and, thus, the push-up heatmap 106A is blank.

Turning to FIG. 1B, a diagram is presented in which further image data is processed to update the prediction as to the user activity being performed in the image data. According to one or more embodiments, the image data 100B may be captured by an electronic device after image data 100A of FIG. 1A. That is, the image data 100B may be a different frame of image data than 100A and may be the next consecutive frame after image data 100A, or a frame captured at some other time after image data 100A. As such, image data 100A and image data 100B may or may not be consecutively captured frames.

In one or more embodiments, image data 100B may be applied to the network trained to predict a body pose present in the image. In the image data 100B, a person is shown in body pose 110B, performing a deeper bend of the knee than that depicted in image data 100A. As described above, a same or different network may be trained to predict future achievable results from a current pose. In some embodiments, the network may consider the current body pose, along with body poses from prior frames. The network may be trained for a predefined set of activities, such as exercises or other repeatable activities. Confidence values are determined for each of these activities based on the current pose and/or prior pose data. The confidence values indicate a likelihood of an outcome for each of the set of activities.

For purposes of the example of FIG. 1B, the same four potential exercises are considered by the network. These include a squat, a lunge, a push-up, and a burpee. For each activity, the set of confidence values for potential outcomes are revised from FIG. 1A, and are plotted to revise a heatmap. In this example, confidence values are determined and plotted for a squat heatmap 102B, a lunge heatmap 104B, a push-up heatmap 106B, and a burpee heatmap 108B. Each of the diagrams depicts a graph in which the x-axis indicates a frame or time at which the activity is predicted to be initiated. The y-axis indicates a predicted duration of the activity. Thus, the confidence values of squat heatmap 102A indicate the likelihood for each potential outcome of the squat (e.g., each potential initiation and duration). In the example of FIG. 1B, squat heatmap 102B depicts the most defined heatmap, and has become even more defined than heatmap 102A from FIG. 1A. In this example, the squat heatmap 102B includes a peak at 20 along the x-axis, and 10 along the y-axis, indicating that the most likely outcome of the motion, among potential outcomes having a squat, is a squat that begins at the 20^{th} frame or otherwise at a timestamp or other measurement at 20, and will last for 10 frames. Meanwhile, the burpee heatmap 108B has become less defined than burpee heatmap 108B. Lunge heatmap 104B and push-up heatmap 106B have also become less defined than lunge heatmap 104A and push-up heatmap 106A, respectively. Notably, while the confidence values plotted in FIG. 1A is predicted using image data 100A, the confidence values plotted in FIG. 1B may be predicted using both the image data 100A and the image data 100B.

According to one or more embodiments, the confidence values (i.e., the heatmaps) for each classification of exercise are monitored to determine whether a stability threshold is satisfied. The stability threshold may be satisfied, for example, if a peak of the heatmap remains stable over a predefined time period, such as a number of frames. As such, a stability threshold may be satisfied, for example, based on confidence values for a single activity and without regard for other activities. In doing so, the embodiments described herein support a technique for predicting multiple activities for a single repetition. As such, the techniques described herein can identify compound exercises, such as a burpee with push-up, or the like. Alternatively, whether the stability threshold is met may be determined by comparing the predictions for different activities. For example, the stability threshold may include determining that a measured level of stability for a particular activity is sufficiently greater than a measured level of stability for the remaining activities. According to some embodiments, the confidence values may be biased toward motion classes based on stability threshold.

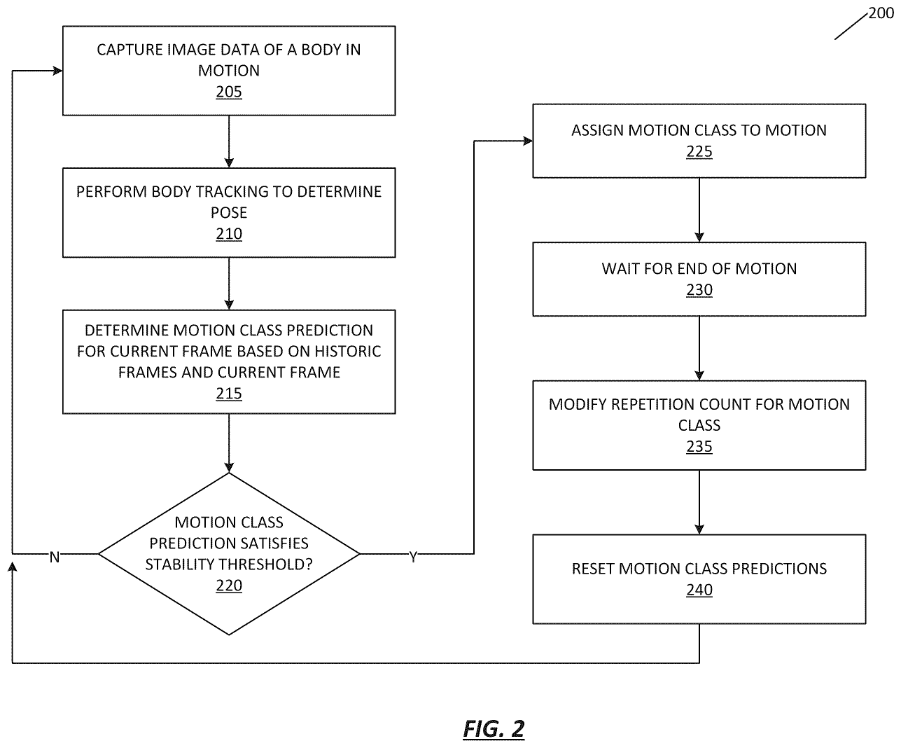

FIG. 2 shows, in flowchart form, a technique for counting repetitions of predicted user activities, according to one or more embodiments. For purposes of explanation, the following steps will be described in the context of particular components. However, it should be understood that the various actions may be taken by alternate components. As an example, a single system may perform all the actions described with respect to FIG. 2. Alternatively, separate components may perform the functions and the functionality may be distributed across multiple systems or devices. In addition, the various actions may be performed in a different order. Further, some actions may be performed simultaneously, and some may not be required, or others may be added.

The flowchart 200 begins at block 205, where image data is captured of a body in motion. According to one or more embodiments, the body may be a user or other person in an environment for which image data and/or other sensor data is collected. According to one or more embodiments, the image data 100A may be captured by an electronic device. Electronic device may be any kind of device that includes a camera or other sensors from which pose information can be detected for a person in the invention. The electronic device capturing the image data may be the same or different from an electronic device performing the prediction of the activity.

The flowchart 200 continues to block 210, where body tracking is performed to determine a body pose. In some embodiments, body tracking is performed by an algorithm taking the image data and/or other sensor data of the user in motion, and predict a pose of the user, either in 2D or 3D. The pose may include, for example, a classification of a pose, a geometric representation of the pose, or the like. As an example, the pose may include a representation of joints and/or segments of a skeleton of a user.

At block 215, a motion class prediction is determined for the current frame based on both the current frame and one or more prior frames (if available). In one or more embodiments, the motion class prediction provides an indication for each of one or more motion classes that the motion is being performed in the frame. The motion class may include, for example, a physical motion, such as an exercise, which is detectable based on pose data by a trained network. That is, a network may be trained to generate prediction data for one or more motion classes indicating a likelihood of one or more potential characteristics or outcomes for the corresponding motion class. That is, in some embodiments, the motion class prediction provides a prediction, during the motion, of a characteristic of the full motion.

The flowchart 200 proceeds to block 220, where a determination is made as to whether a motion class prediction satisfies a stability threshold. That is, a motion class prediction may be updated for each frame of a set of frames capturing the motion. The one or more predicted values are compared over time to determine whether the predicted values for the particular motion class have stabilized. The stability threshold may be satisfied, for example, if a peak of a heatmap remains stable over a predefined time period, as described above with respect to FIG. 1B. As another example, the stability threshold may be determined to be met by comparing the predictions for different activities. If a determination is made that the motion class prediction has not satisfied a stability threshold, then the flowchart returns to block 205, and the system continues to capture image data of the body in motion and determine motion class predictions.

Returning to block 220, if a determination is made that the motion class prediction satisfies a stability threshold, then the flowchart proceeds to block 225 and the motion class is assigned to the motion. In some embodiments, the system may continue to generate motion class predictions for other motion classes. For example, the system can independently track class predictions such that two or more motion classes can be identified. As such, the system supports a technique for predicting multiple motion types for a single motion, such as if the person is performing a compound motion comprising two different motion classes. Alternatively, the system may cease determining motion class predictions once the motion class prediction for one of the motion classes satisfies the stability threshold.

Upon assigning the motion class, the flowchart 200 proceeds to block 230 where the system waits for the motion to be complete. In some embodiments, the system may rely on data from the prediction, such as a duration, to predict the end of the motion. Additionally, or alternatively, the system may confirm the motion is complete using the image data or pose data against the expected pose for the end of the motion. Upon detecting the end of the motion, the predicted motion class can be confirmed.

The flowchart 200 proceeds to block 235 where, in response to the end of the motion, a repetition count for the motion class is modified. A repetition count may be tracked for the different motion classes. According to one or more embodiments, the system may begin tracking a repetition count upon determining a completion of a particular motion or a series of motion classes which are repeated. That is, the repetition count may be associated with individual motion classes, or may be associated with a series of motion classes. In some embodiments, the techniques described herein may be used by an application configured to track repetitive activity, such as exercise. In doing so, a repetition count may be modified, such as incremented, at the end of the motion. In some embodiments, the repetition count may be modified for each motion class identified to satisfy the stability threshold at block 220 in a situation in which multiple motion classes are detected within a single motion. In some embodiments, if multiple motion classes are detected, a single repetition count will be presented to the user representative of a compound activity comprising the detected motion classes. The single repetition count will be incremented or otherwise modified at the end of the last motion of the multiple motions.

The flowchart then concludes at block 240, where the motion class predictions are reset. For example, predicted value for each motion class are cleared such that new prediction values can be determined for a next repetition of the motion. According to one or more embodiments, the prediction network may be biased toward generating prediction values based on the previously-predicted motion class or classes. As such, after a repetition ends, prediction values for the prior-predicted motion class may soon increase. The flowchart then returns to block 205, where the system continues to capture image data of a body in motion to generate prediction values for the next repetition or motion.

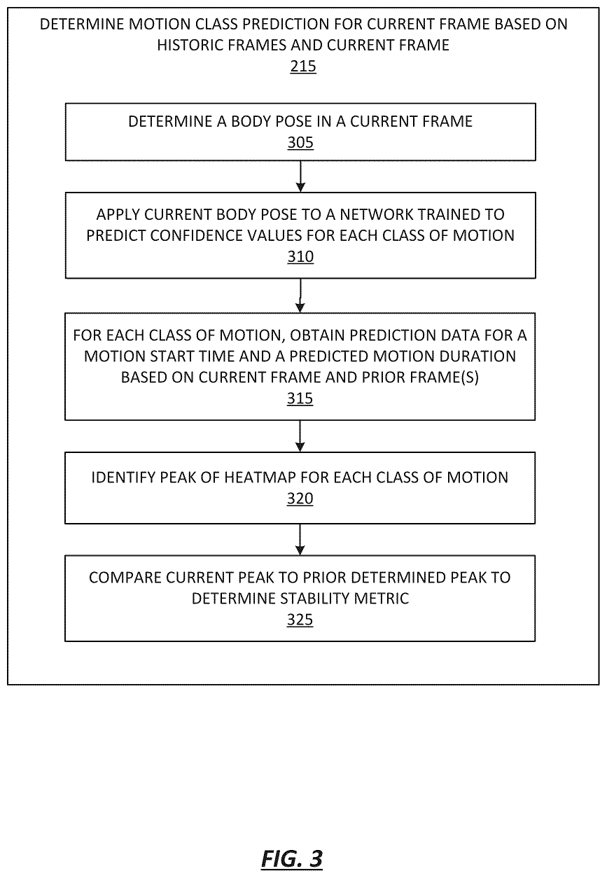

According to one or more embodiments, the prediction values correspond to confidence values for characteristics of multiple potential outcomes for a given motion class, using a current frame and prior frames. The prediction network may be trained to provide confidence values for a single motion class or multiple motion classes simultaneously. FIG. 3 shows, in flowchart form, a technique for determining confidence values for motion classes, in accordance with one or more embodiments. In particular, FIG. 3 depicts an example detailed flow for determining motion class predictions as described above with respect to block 215 of FIG. 2. For purposes of explanation, the following steps will be described in the context of particular components. However, it should be understood that the various actions may be taken by alternate components. As an example, a single system may perform all the actions described with respect to FIG. 3. Alternatively, separate components may perform the functions and the functionality may be distributed across multiple systems or devices. In addition, the various actions may be performed in a different order. Further, some actions may be performed simultaneously, and some may not be required, or others may be added.

The flowchart begins at block 305, where a body pose is determined in a current frame. In some embodiments, body tracking is performed by an algorithm taking the image data and/or other sensor data of the user in motion, and predict a pose of the user, either in 2D or 3D. The pose may include, for example, a classification of a pose, a geometric representation of the pose, or the like. As an example, the pose may include a representation of joints and/or segments of a skeleton of a user. In some embodiments, a pose may determined for each frame, or a subset of the frames capturing the motion.

At block 310, the body pose for a current frame is applied to a network trained to predict confidence values for each class of motion. According to one or more embodiments, the network is trained to predict a likelihood of multiple potential characteristics of outcomes for each motion class, as shown at block 315. In some embodiments, for each motion class for which the network is trained, a set of confidence values may be determined indicating a likelihood of different characteristics of the completed version of that motion class in accordance with a current frame and, if available, one or more prior frames captured prior to completion of the motion. As an example, as described above with respect to FIG. 1, a set of confidence values are determined for a set of initiation and duration characteristics for each exercise. According to one or more embodiments, the confidence values for the potential outcomes for each motion class are incorporated into a graph such that a heatmap of the confidence values are formed.

The flowchart proceeds to block 320, where a peak of the heatmap is determined based on the confidence values. In some embodiments, the peak of the heatmap may be determined for each motion class for which the network predicts confidence values. The peak of the heatmap may indicate a point in a coordinate system associated with the outcome characteristics indicating a most likely outcome in accordance with the predicted values. As new frames are processed, the predictions may change and the heatmap may shift. As such, the flowchart concludes at block 325, where the system compares a current peak to prior determined peaks to determine a stability metric. That is, as new frames come in, if a most likely potential outcome for a given motion class remains the same or substantially the same (for example, within a predefined threshold), then the motion class and, optionally, the characteristics associated with the stable peak, can be assigned to the motion.

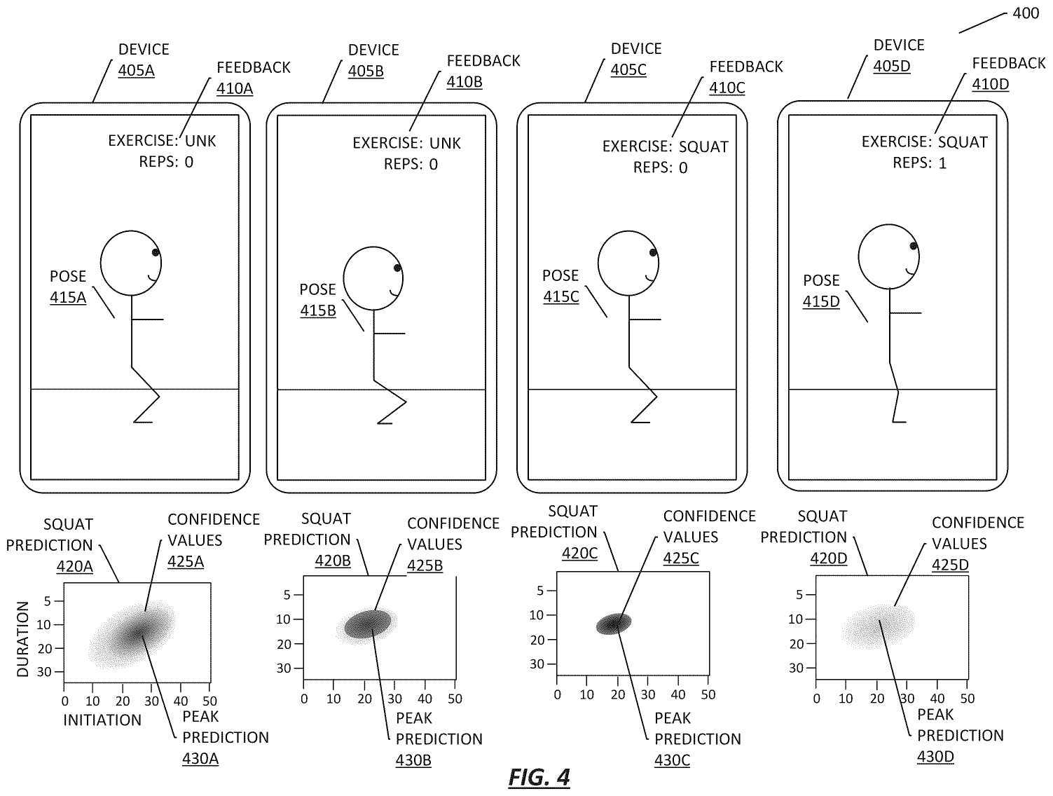

FIG. 4 shows a diagram of a technique for counting repetitions of predicted user activities, in flowchart form, a technique for applying a visual treatment to image content, according to one or more embodiments. It should be understood that the particular embodiment shown is merely used as an example for explanation of certain embodiments and is not particularly intended to limit the scope.

In the example diagram 400, four device instances 405A-D are depicted indicating a view of a user interface on the device 405 at four different points in time. Device instance 405A shows a view of the interface on the device at a first time. In device instance 405A, an image frame of a person is captured. The image may be processed to determine a pose 415A. The pose 415A may be fed into a network to obtain one or more predictions for one or more motion classes. For purposes of the example 400, prediction values are determined for a single motion class. In this case, the prediction values correspond to a squat prediction 420A. However, it should be understood that multiple sets of predictions could be obtained for multiple motion classes, as described above with respect to FIG. 1.

In this instance, the confidence values 425A represent a set of confidence values associated with different outcome characteristics for the motion being performed by the person performing the pose 415A. In this example, a confidence value is determined for each potential outcome having different initiation metrics and duration metrics, indicating, along the x-axis, a measure of the initiation of the motion class (e.g., the squat) and, along the y-axis, a duration of the motion. A peak prediction 430A may be determined. In some embodiments, the peak prediction 430A may be used to determine whether the peak has stabilized yet. The peak may stabilize if the coordinates associated with the peak stay the same or within a margin of error for some predetermined about of time or frames. For purposes of this example, the peak is considered to not have stabilized yet at device instance 405A.

In some embodiments, the user interface may show user feedback 410A regarding the predicted motion and/or repetitions. Here, because the peak prediction 430A has not yet been determined to be stabilized, the exercise is not yet listed. Similarly, no repetitions of any action have been detected.

At a next time, device instance 405B shows a view of the interface on the device at a second time. In device instance 405B, a second image frame of the person is captured. In some embodiments, the second frame may be captured immediately consecutively after the first frame. Alternatively, the second frame shown at device instance 405B may be captured at some time after the first frame shown at device instance 405A. The second image may be processed to determine a pose 415B. The pose 415B may be fed into a network, along with the pose from prior frames, such as pose 415A, to obtain one or more predictions for one or more motion classes. For purposes of the example 400, prediction values are determined for a single motion class.

In this instance, the confidence values 425B represent a set of confidence values associated with different outcome characteristics for the motion being performed by the person performing the pose 415B, with consideration of the pose 415A and/or confidence values 425A. In this example, a confidence value is determined for each potential outcome having different initiation metrics and duration metrics, indicating, along the x-axis, a measure of the initiation of the motion class (e.g., the squat) and, along the y-axis, a duration of the motion. An updated peak prediction 430B may be determined. In some embodiments, the peak prediction 430B may be used to determine whether the peak has stabilized yet. The peak may stabilize if the coordinates associated with the peak stay the same or within a margin of error for some predetermined about of time or frames. Here, the peak prediction 430B may be compared to peak prediction 430A. For example, a determination may be made as to where the peak prediction 430B is the same or within a threshold margin of error of peak prediction 430A. For purposes of this example, the peak prediction is determined to not have yet stabilized at device instance 405B.

In some embodiments, the user interface may show user feedback 410B regarding the predicted motion and/or repetitions. Here, because the peak prediction 430B has not yet been determined to be stabilized, the exercise is not yet listed. Similarly, no repetitions of any action have been detected.

Moving on to a third time, device instance 405C shows a view of the interface on the device sometime after the view of the interface on device instance 405B. In device instance 405C, a third image frame of the person is captured. In some embodiments, the third frame may be captured immediately consecutively after the second frame. Alternatively, the third frame shown at device instance 405C may be captured at some time after the second frame shown at device instance 405B. The third image may be processed to determine a pose 415C. The pose 415C may be fed into a network, along with the pose from prior frames, such as pose 415A and pose 415B, to obtain one or more predictions for one or more motion classes. For purposes of the example 400, prediction values are determined for a single motion class.

In this instance, the confidence values 425C represent a set of confidence values associated with different outcome characteristics for the motion being performed by the person performing the pose 415C, with consideration of the pose 415A and pose 415B, and/or confidence values 425A and 425B. In this example, a confidence value is determined for each potential outcome having different initiation metrics and duration metrics, indicating, along the x-axis, a measure of the initiation of the motion class (e.g., the squat) and, along the y-axis, a duration of the motion. An updated peak prediction 430B and/or 430C may be determined. In some embodiments, the peak prediction 430C may be used to determine whether the peak has stabilized yet. Here, the peak prediction 430C may be compared to peak prediction 430A and/or peak prediction 430C. For purposes of this example, the peak prediction is determined to have stabilized, as it is substantially similar to peak prediction 430B.

In some embodiments, the user interface may show user feedback 410C regarding the predicted motion and/or repetitions. Here, because the peak prediction 430C has stabilized, the system has determined that a squat is being performed. However, because the system predicts the motion while it is in progress, the squat has not yet been completed and, thus, no repetitions of any action have been detected.

Finally, at a fourth time, device instance 405D shows a view of the interface on the device sometime after the view of the interface on device instance 405C. In device instance 405D, a fourth image frame of the person is captured. In some embodiments, the fourth frame may be captured immediately consecutively after the third frame. Alternatively, the fourth frame shown at device instance 405D may be captured at some time after the third frame shown at device instance 405C. The fourth image may be processed to determine a pose 415D. The pose 415D may be fed into a network, along with the pose from prior frames, such as pose 415A, pose 415B, and pose 415C, to obtain one or more predictions for one or more motion classes. For purposes of the example 400, prediction values are determined for a single motion class.

In this instance, the squat which was previously predicted has now been determined to be compete. As such, the prior confidence values 425C are reset and no longer shown in squat prediction 420D. However, in some embodiments, the network is trained to show bias toward multiple repetitions of a motion class. As such, upon the conclusion of a motion, the peak prediction 430D for a next motion may quickly begin showing confidence values 425D for the squat prediction 420D.

In some embodiments, the user interface may show user feedback 410D regarding the predicted motion and/or repetitions. Here, because the motion was previously determined to be a squat, the feedback 410D indicates that the current exercise is a squat. Further, because the motion has been determined to be complete, the feedback 410 additionally shows a modified repetition count, showing 1 squat being complete.

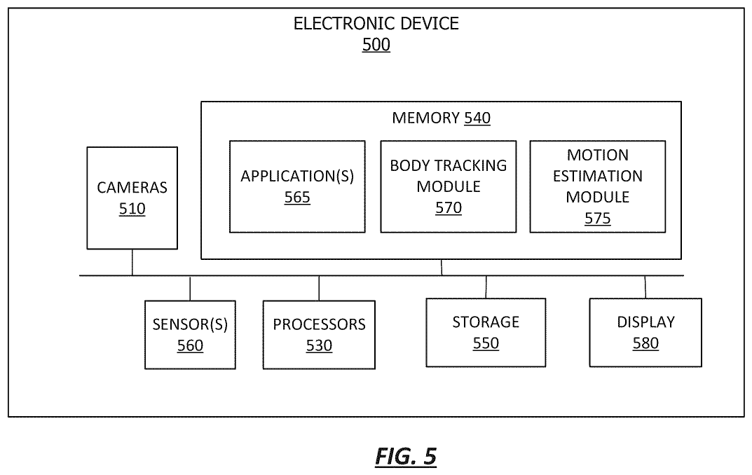

Referring to FIG. 5, a simplified block diagram of an electronic device 500 is depicted, in accordance with one or more embodiments of the disclosure. Electronic device 500 may be part of a multifunctional device, such as a mobile phone, tablet computer, personal digital assistant, portable music/video player, wearable device, or any other electronic device that includes a camera system. FIG. 5 shows, in block diagram form, an overall view of a system diagram capable of supporting proximity detection and breakthrough, according to one or more embodiments. Electronic device 500 may be connected to other network devices across a network via network interface, such as mobile devices, tablet devices, desktop devices, as well as network storage devices such as servers and the like. In some embodiments, electronic device 500 may communicably connect to other electronic devices via local networks to share sensor data and other information.

Electronic Device 500 may include one or more processors 530, such as a central processing unit (CPU). Processor 530 may be a system-on-chip such as those found in mobile devices and include one or more dedicated graphics processing units (GPUs). Further, processor 530 may include multiple processors of the same or different type. Electronic Device 500 may also include a memory 540. Memory 540 may include one or more different types of memory, which may be used for performing device functions in conjunction with processor 530. For example, memory 540 may include cache, ROM, and/or RAM. Memory 540 may store various programming modules during execution, including applications module 565, body tracking module 570, and motion estimation module 575. According to some embodiments, application(s) 565 may provide a user with activity-based tracking and feedback. As an example, application(s) may include health applications, exercise applications, or other application where predicting and tracking user activity is utilized. Body tracking module 570 may utilize data from camera(s) 510 and/or sensor(s) 560, such as proximity sensors, to collect sensor data of a person performing a motion or activity, from which body pose can be derived. For example, body tracking module 570 may utilize a body tracking pipeline to predict a skeleton or other representation of a body in image data. Motion estimation module 575 may include functionality for utilizing the body tracking data to predict a current activity being performed by a person based on pose information over a series of frames, and in real time prior to the completion of the activity. Motion estimation module may utilize a network trained to generate predictions for characteristics of outcomes of one or more activities based on a current pose and prior pose information. In some embodiments, motion estimation module 575 may provide feedback related to the predicted activities, for example on a user interface presented to a user on a display 580 of the electronic device. The electronic device may include one or more storage devices 550, which may be used to hold data to facilitate processing of application(s) 565, body tracking module 570, and/or motion tracking module 575.

Electronic device 500 may include one or more cameras 510. The camera(s) 510 may each include an image sensor, a lens stack, and other components that may be used to capture images. In one or more embodiments, the cameras may be directed in different directions in the electronic device. For example, a front-facing camera may be positioned in or on a first surface of the electronic device 500, while the back-facing camera may be positioned in or on a second surface of the electronic device 500. In some embodiments, camera(s) 510 may include one or more types of cameras, such as RGB cameras, depth cameras, and the like. Electronic device 500 may include one or more sensor(s) 560 which may be used to detect physical obstructions in an environment. Examples of the senor(s) 560 include LIDAR and the like.

In one or more embodiments, the electronic device 500 may also include a display 580. Display 580 may be any kind of display device, such as an LCD (liquid crystal display), LED (light-emitting diode) display, OLED (organic light-emitting diode) display, or the like. In addition, display 580 could be a semi-opaque display, such as a heads-up display, pass-through display, or the like. Display 580 may present content in association with application(s) 565.

Although electronic device 500 is depicted as comprising the numerous components described above, in one or more embodiments, the various components may be distributed across multiple devices. Further, additional components may be used and/or some combination of the functionality of any of the components may be combined.

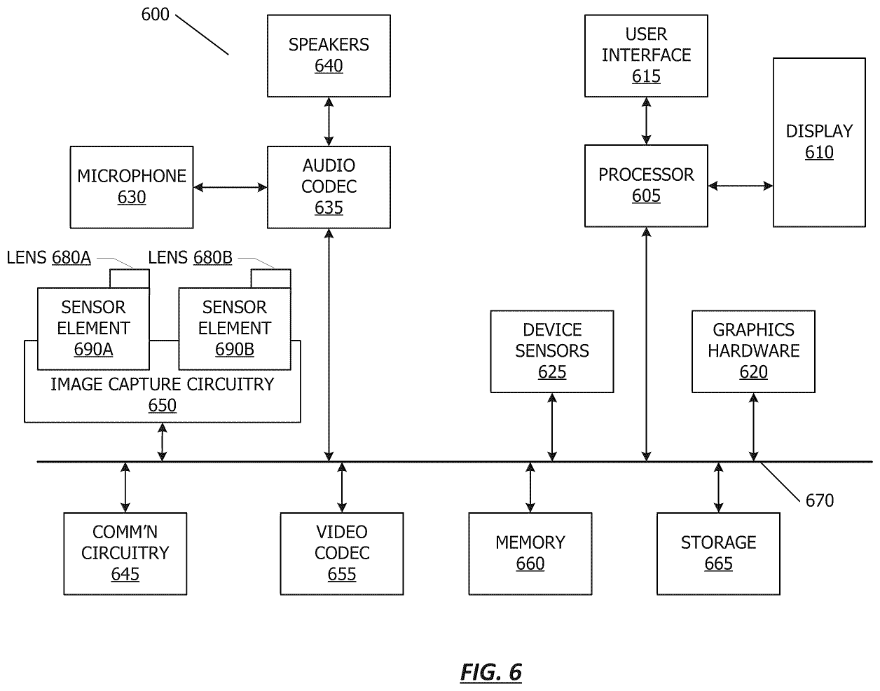

Referring now to FIG. 6, a simplified functional block diagram of illustrative multifunction device 600 is shown according to one embodiment. Multifunction electronic device 600 may include processor 605, display 610, user interface 615, graphics hardware 620, sensors 625 (e.g., proximity sensor/ambient light sensor, accelerometer and/or gyroscope), microphone 630, audio codec(s) 635, speaker(s) 640, communications circuitry 645, digital image capture circuitry 650 (e.g., including camera system), video codec(s) 655 (e.g., in support of digital image capture unit), memory 660, storage device 665, and communications bus 670. Multifunction electronic device 600 may be, for example, a digital camera or a personal electronic device such as a personal media player, mobile telephone, head-mounted device, or a tablet computer.

Processor 605 may execute instructions necessary to carry out or control the operation of many functions performed by device 600 (e.g., the generation and/or processing of images as disclosed herein). Processor 605 may, for instance, drive display 610 and receive user input from user interface 615. User interface 615 may allow a user to interact with device 600. For example, user interface 615 can take a variety of forms, such as a button, keypad, dial, a click wheel, keyboard, display screen and/or a touch screen. Processor 605 may also, for example, be a system-on-chip such as those found in mobile devices and include a dedicated graphics processing unit (GPU). Processor 605 may be based on reduced instruction-set computer (RISC) or complex instruction-set computer (CISC) architectures or any other suitable architecture and may include one or more processing cores. Graphics hardware 620 may be special purpose computational hardware for processing graphics and/or assisting processor 605 to process graphics information. In one embodiment, graphics hardware 620 may include a programmable GPU.

Image capture circuitry 650 may include two (or more) lens assemblies 680A and 680B, where each lens assembly may have a separate focal length. For example, lens assembly 680A may have a short focal length relative to the focal length of lens assembly 680B. Each lens assembly may have a separate associated sensor element 690. Alternatively, two or more lens assemblies may share a common sensor element. Image capture circuitry 650 may capture still and/or video images. Output from image capture circuitry 650 may be processed, at least in part, by video codec(s) 655, and/or processor 605, and/or graphics hardware 620, and/or a dedicated image processing unit or pipeline incorporated within circuitry 650. Images so captured may be stored in memory 660 and/or storage 665.

Sensor and camera circuitry 650 may capture still and video images that may be processed in accordance with this disclosure, at least in part, by video codec(s) 655, and/or processor 605, and/or graphics hardware 620, and/or a dedicated image processing unit incorporated within circuitry 650. Images so captured may be stored in memory 660 and/or storage 665. Memory 660 may include one or more different types of media used by processor 605 and graphics hardware 620 to perform device functions. For example, memory 660 may include memory cache, read-only memory (ROM), and/or random access memory (RAM). Storage 665 may store media (e.g., audio, image, and video files), computer program instructions or software, preference information, device profile information, and any other suitable data. Storage 665 may include one more non-transitory computer-readable storage mediums including, for example, magnetic disks (fixed, floppy, and removable) and tape, optical media such as CD-ROMs and digital video disks (DVDs), and semiconductor memory devices such as Electrically Programmable Read-Only Memory (EPROM), and Electrically Erasable Programmable Read-Only Memory (EEPROM). Memory 660 and storage 665 may be used to tangibly retain computer program instructions or code organized into one or more modules and written in any desired computer programming language. When executed by, for example, processor 605, such computer program code may implement one or more of the methods described herein.

The scope of the disclosed subject matter should be determined with reference to the appended claims, along with the full scope of equivalents to which such claims are entitled. In the appended claims, the terms “including” and “in which” are used as the plain-English equivalents of the respective terms “comprising” and “wherein.”

1. A non-transitory computer readable medium comprising computer readable code executable by one or more processors to:
capture image data of a body in motion;
determine, based on a first set of frames of the image data, one or more predictions for one or more motion classes;
in response to receiving an additional frame of the image data, revise the one or more predictions for the one or more motion classes; and
in response to determining that the one or more predictions for at least one of the one or more motion classes satisfies a stability threshold, assign the at least one of the one or more motion classes to the body in motion.

2. The non-transitory computer readable medium of claim 1, wherein the one or more predictions comprises, for each motion class, a confidence value for each of a set of potential durations for a corresponding motion class.

3. The non-transitory computer readable medium of claim 2, wherein the motion is a repeated motion, and further comprising computer readable code to:
determine that a repetition of the at least one of the one or more motion classes has ended; and
in response to determining that the repetition has ended, modify a repetition count for the at least one of the one or more motion classes.

4. The non-transitory computer readable medium of claim 3, wherein the determination that the repetition has ended is based on a predicted duration of the one or more predictions that satisfies the stability threshold for the motion class.

5. The non-transitory computer readable medium of claim 4, wherein the computer readable code to determine the confidence values for the one or more classes comprises computer readable code to:
predict, based on one or more body poses captured in the first set of frames, an initialization for the motion class,
wherein the determination that the repetition has ended is further based on the predicted initialization for the motion class.

6. The non-transitory computer readable medium of claim 3, further comprising computer readable code to, in response to determining that the repetition has ended:
reset the confidence values for each of the one or more motion classes.

7. The non-transitory computer readable medium of claim 6, further comprising computer readable code to:
capture additional image data of a body in motion comprising a second set of frames captured subsequent to the first set of frames; and
determine, based on the second set of frames of the image data, one or more updated confidence values for one or more motion classes,
wherein the one or more updated confidence values are determined in accordance with a bias toward the at least one of the one or more motion classes based on the confidence values for the at least one of the one or more motion classes satisfying the stability threshold.

8. A method comprising:
capturing image data of a body in motion;
determining, based on a first set of frames of the image data, one or more predictions for one or more motion classes;
in response to receiving an additional frame of the image data, revising the one or more predictions for the one or more motion classes; and
in response to determining that the one or more predictions for at least one of the one or more motion classes satisfies a stability threshold, assign the at least one of the one or more motion classes to the body in motion.

9. The method of claim 8, wherein the one or more predictions comprises, for each motion class, a confidence value for each of a set of potential durations for a corresponding motion class.

10. The method of claim 9, wherein the motion is a repeated motion, and further comprising:
determining that a repetition of the at least one of the one or more motion classes has ended; and
in response to determining that the repetition has ended, modifying a repetition count for the at least one of the one or more motion classes.

11. The method of claim 10, wherein the determination that the repetition has ended is based on a predicted duration of the one or more predictions that satisfies the stability threshold for the motion class.

12. The method of claim 11, wherein determining the confidence values for the one or more motion classes comprise:
predicting, based on one or more body poses captured in the first set of frames, an initialization for the motion class,
wherein the determination that the repetition has ended is further based on the predicted initialization for the motion class.

13. The method of claim 10, further comprising, in response to determining that the repetition has ended:
resetting the confidence values for each of the one or more motion classes.

14. The method of claim 13, further comprising:
capturing additional image data of a body in motion comprising a second set of frames captured subsequent to the first set of frames; and
determining, based on the second set of frames of the image data, one or more updated confidence values for one or more motion classes,
wherein the one or more updated confidence values are determined in accordance with a bias toward the at least one of the one or more motion classes based on the confidence values for the at least one of the one or more motion classes satisfying the stability threshold.

15. A system comprising:
one or more processors; and
one or more computer readable media comprising computer readable code executable by the one or more processors to:
capture image data of a body in motion;
determine, based on a first set of frames of the image data, one or more predictions for one or more motion classes;
in response to receiving an additional frame of the image data, revise the one or more predictions for the one or more motion classes; and
in response to determining that the one or more predictions for at least one of the one or more motion classes satisfies a stability threshold, assign the at least one of the one or more motion classes to the body in motion.

16. The system of claim 15, wherein the one or more predictions comprises, for each motion class, a confidence value for each of a set of potential durations for a corresponding motion class.

17. The system of claim 16, wherein the motion is a repeated motion, and further comprising computer readable code to:
determine that a repetition of the at least one of the one or more motion classes has ended; and
in response to determining that the repetition has ended, modify a repetition count for the at least one of the one or more motion classes.

18. The system of claim 17, wherein the determination that the repetition has ended is based on a predicted duration of the one or more predictions that satisfies the stability threshold for the motion class.

19. The system of claim 18, wherein the computer readable code to determine the confidence values for the one or more classes comprises computer readable code to:
predict, based on one or more body poses captured in the first set of frames, an initialization for the motion class,
wherein the determination that the repetition has ended is further based on the predicted initialization for the motion class.

20. The system of claim 17, further comprising computer readable code to, in response to determining that the repetition has ended:
reset the confidence values for each of the one or more motion classes.
